(19) United States
(12) Patent Application Publication
(10) Pub. No.: US 2009/0030434 A1
(43) Pub. Date: Jan. 29, 2009

(54) Surgical Fasteners and Devices for Surgical Fasteners

(75) Inventors: Adrian Paz, Petach Tikva (IL); Gilad Heftman, Kibbutz Ein Gev (IL); Eitan Hod, Zichron Ya'akov (IL)

Correspondence Address: THE NATH LAW GROUP, 112 South West Street, Alexandria, VA, 22314, US

(73) Assignee: ENDOGUN MEDICAL SYSTEMS LTD., Kibbutz HaOgen (IL)

(21) Appl. No.: 11/990,262

(22) PCT Filed: Aug. 9, 2006
(86) PCT No.: PCT/IL2006/000917

§ 371 Date: Jun. 18, 2008

Related U.S. Application Data
(60) Provisional application No. 60/706,907, filed on Aug. 11, 2005.

(51) Int. Cl. A61B 17/03

(52) U.S. Cl. 606/151; 606/1

(57) ABSTRACT

The invention provides fasteners for surgical fastening. The surgical fastener of the invention has a crown from which extend two or more prongs. The prongs are parallel to the axis of the crown and each prong is provided with one or more barbs. The invention also provides a system for surgical fastening device configured to deploy one or more surgical fasteners of the invention and one or more surgical fasteners of the invention.

### FIELD OF THE INVENTION

This invention relates to surgical fasteners and to surgical fastening devices.

### BACKGROUND OF THE INVENTION

Surgical fasteners are used instead of surgical suturing, which is often both time consuming and inconvenient, in order to join two tissue locations or to attach a surgical material, such as a surgical mesh or filament, to a tissue surface. A surgeon can often use a stapling apparatus to implant a fastener into a body tissue and thus accomplish in a few seconds, what would take a much longer time to suture. A surgical fastener has been used, for example in inguinal hernia surgery to fasten polypropylene mesh to the abdominal wall in order to reinforce the abdominal wall.

A variety of surgical fasteners and fastening devices are available for endoscopic or open procedures, to attach tissues together, or to attach a mesh patch to a tissue. One such surgical fastener is a surgical stapler, or clip applicator. In this device, a plurality or stack of unformed staples are contained within a cartridge and are sequentially advanced or fed within the instrument by a spring mechanism. A secondary feeding mechanism is employed to separate the distal most staple from the stack, and to feed the distal-most stapler into the staple deployment mechanism.

In some applications, the body tissue is accessible from two opposite direction so that an anvil may be used to deform the prongs of a staple after having passed through the body tissue. In applications where access to the tissue is from only one direction, an anvil may be used to deform the crown of the staple so that the prongs project towards each other in the body tissue so as to hold the staple in the tissue.

Another stapler mechanism, used mostly for mesh attachment to tissue does not use an anvil. Instead, a fastener comprising a helical wire is screwed or rotated into a tissue, in order to join tissues to affix a polypropylene or similar material mesh or other patch to the tissue together. Instruments and fasteners of this type are found in U.S. Pat. No. 5,582,616, U.S. Pat. No. 5,810,882, and U.S. Pat. No. 5,830,221. Another type of fastener that does not need an anvil applies fasteners made from a shape memory alloy such as Nitinol™. These fasteners are mainly used to fasten prosthetic material or artificial mesh to tissue.

### SUMMARY OF THE INVENTION

In its first aspect, the present invention provides a surgical fastener. The fastener of the invention has a crown from which extend two or more prongs. The prongs are parallel to each other and in general are perpendicular to the crown. Each prong is provided with one or more barbs that point towards the crown and away from the prong tip.

The fastener is inserted into a body tissue with neither the crown nor the prongs being deformed. In particular, the prongs remain straight and parallel to each other after insertion of the fastener into the tissue. After deployment, the crown remains at the surface of the tissue, and does not penetrate into the tissue. The prongs are preferably in the range of 3 to 7 mm in length so that after deployment, the barbs are located in the fascia layer. Collagen fibers in the fascia layer entangle the barbs and thus promote the immobilization of the fastener in the tissue. The surgical fasteners of the invention are preferably manufactured from a biocompatible material such as stainless steel, nitinol, titanium plastic materials, and may be manufactured from a biodegradable material such as PLA, PLGA, poly-caprolactone, polydiaxone etc.

FIG. 2 shows deployment of the fastener 1. As shown in FIG. 2a, the fastener 1 is to be deployed at the surface 18 of a body tissue 16. A surgical material 20 that may have a solid or mesh-like structure is first applied to the tissue surface 18. The surgical material may have any shape such as an elongated strip or a rectangular patch. Using a deployment device described in detail below, the fastener 1 is inserted through the surgical material 20 and the surface 18 into the tissue 16, as shown in FIG. 2b.

In its second aspect, the present invention provides a surgical fastening device for deploying one or more surgical fasteners of the invention. In a preferred embodiment of the surgical fastening device of the invention, a hollow cylindrical shaft extends from a handle portion that stores one or more surgical fasteners of the invention. An actuating mechanism contained in the handle portion sequentially ejects the fasteners from the distal end of the shaft.

In a most preferred embodiment of the surgical fastener, the distal end of the shaft is provided with one or more projections configured to engage a surgical mesh material. A piece of a surgical mesh material is engaged onto the projections at a first region of the mesh material. The distal end of the shaft with the surgical mesh material engaged on it is then delivered to a first location on a tissue surface. The actuating mechanism of the device is then actuated to eject a fastener from the distal end of the shaft into the tissue at the first location. As the fastener is ejected from the distal end of the shaft, the prongs of the fastener pass through the mesh so as to pin the mesh to the tissue at the first location.

The distal end of the shaft is then disengaged from the mesh. The projections on the distal end of the shaft are then engaged at a second region of the mesh. The distal end of the shaft is then brought to a second location where the mesh is to be pinned. The actuating mechanism of the device is actuated again to eject a second fastener at the second location to pin the second region of the mesh at the second location in the body. The surgical fastening device is then disengaged from the mesh and removed from the body, leaving the first and second body locations joined by the mesh. The same procedure may be repeated to pin the mesh or surgical material at additional locations to the tissue.

In its third aspect, the invention provides a system comprising a surgical fastening device of the invention and one or more surgical fasteners of the invention. The system of the invention may be used to attach one or more pieces of a surgical material, especially a surgical mesh, at one or more locations on tissue surfaces in a body cavity. For example, the system of the invention may be used in laparoscopic repair of abdominal wall hernia, in which abdominal wall defects are closed using surgical meshes or materials attached to fascia surfaces.

Thus, in its first aspect, the present invention provides a surgical fastener comprising:
(a) a crown having an axis; and
(b) two or more prongs extending from the crown, each prong being parallel to the axis and each prong being provided with one or more barbs.

In its second aspect, the invention provides a system for surgical fastening comprising:
(a) a surgical fastening device configured to deploy one or more surgical fasteners, each surgical fastener comprising:
   (i) a crown having an axis; and
   (ii) two or more prongs extending from the crown, each prong being parallel to the axis and each prong being provided with one or more barbs;
the surgical fastening device comprising:
   a compartment configured to contain the one or more surgical fasteners; and
   an actuating mechanism configured to eject a fastener from the device without deforming the fastener; and
(b) One or more surgical fasteners, each surgical fastener comprising:
   (i) a crown having an axis; and
   (ii) two or more prongs extending from the crown, each prong being parallel to the axis and each prong being provided with one or more barbs.

In its third aspect, the invention provides use of the surgical system of the invention in a surgical procedure.

In its fourth aspect, the invention provides a method for surgical fastening, comprising:
(a) providing a surgical fastening device configured to deploy one or more surgical fasteners, each surgical fastener comprising:
   (i) a crown having an axis; and
   (ii) two or more prongs extending from the crown, each prong
being parallel to the axis and each prong being provided with one or more barbs;
the surgical fastening device comprising:
   a. a compartment containing one or more of the surgical fasteners; and
   b. an actuating mechanism configured to eject a fastener from the device without deforming the fastener;
(b) delivering at least a portion of the surgical fastening device to a body site; and
(c) actuating the actuating mechanism to eject a surgical fastener into a body tissue at the body site.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 shows a surgical fastener in accordance with one embodiment of the invention;

FIG. 2 shows deployment of the surgical fastener of FIG. 1 in a body tissue;

FIG. 3 shows a surgical fastener in accordance with a second embodiment of the invention;

FIG. 4 shows a surgical fastener in accordance with a third embodiment of the invention;

FIG. 5 shows a surgical fastener in accordance with a fourth embodiment of the invention;

FIG. 6 shows a surgical fastener in accordance with a fifth embodiment of the invention;

FIG. 7 shows deployment of the surgical fastener of FIG. 6 in a body tissue;

FIG. 8 shows a surgical fastener in accordance with a sixth embodiment of the invention;

FIG. 9 shows deployment of the surgical fastener of FIG. 8 in a body tissue;

FIG. 10 shows a surgical fastening device in accordance one embodiment of the invention having a cylindrical shaft extending from a handle portion;

FIG. 11 shows a cross-sectional view of the surgical fastening device of FIG. 10;

FIG. 12 shows the distal end of the shaft of the surgical fastening device of FIGS. 10 and 11

FIG. 13 shows an arrangement of surgical fasteners of the invention on an inner sleeve contained in the shaft of the surgical fastening device of FIGS. 10 and 11;

FIG. 14 shows an outer sleeve contained in the shaft of the surgical fastening device of FIGS. 10 and 11;

FIG. 15 shows an arrangement of surgical fasteners of the invention in a shaft of a surgical fastening device of the invention;

FIG. 16 shows another arrangement of surgical fasteners of the invention in a shaft of a surgical fastening device of the invention;

FIG. 17 use of the surgical fastening device of the invention in a laproscopic surgical procedure involving pinning a surgical mesh to a body tissue; and

FIG. 18 shows use of the surgical fastening device of the invention in vaginal repair of stress incontinence.

### DETAILED DESCRIPTION OF THE INVENTION

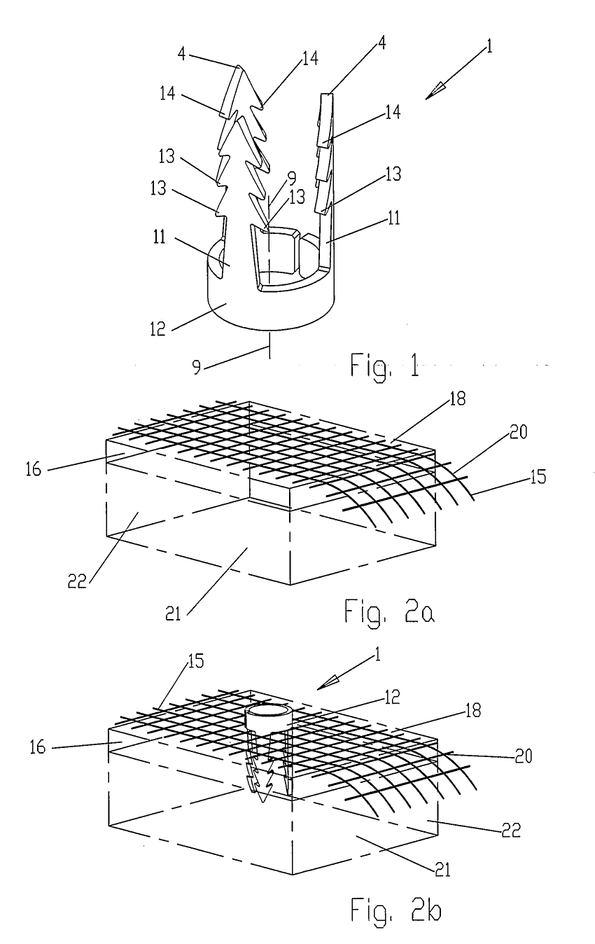

FIG. 1 shows a surgical fastener 1 in accordance with one embodiment of the invention. The fastener 1 has a crown 12 in the form of a hollow cylinder having a longitudinal axis 9. Three prongs 11 extend from the crown 12. This is by way of example, only, and the fastener 1 may have any number of parallel prongs that is at least two. Each of the prongs 11 terminates in a pointed tip 4. The prongs 11 are oriented parallel to the axis 9 of the crown 12. Each prong is provided with one or more barbs 13. In the embodiment of FIG. 1, each prong is provided with three pairs of barbs 13. This is by way of example only, and the prongs may have any number of barbs that is at least one. Each barb 13 is pointed towards the crown 12 and away from the tip 4. The barbs may appear in pairs, as in the embodiment of FIG. 1, with a barb on one edge of a prong being opposite a barb on the other edge of the prong. Alternatively, the barbs on a prong may be staggered, or the barbs may be present only on one edge of a prong.

FIG. 2 shows deployment of the fastener 1. As shown in FIG. 2a, the fastener 1 is to be deployed at the surface 18 of a body tissue 16. The body tissue 16 is indicated in FIG. 2 by broken lines. A surgical material 20 that may have a solid or mesh-like structure is first applied to the tissue surface 18. The surgical material may have any shape such as an elongated strip or a rectangular patch. Using a deployment device described in detail below, the fastener 1 is inserted through the surgical material 20 and the surface 18 into the tissue 16, as shown in FIG. 2b. During insertion of the fastener 1 into the tissue 16, neither the crown 12 or the prongs 11 is deformed. In particular, the prongs 11 remain straight and parallel to each other after insertion of the fastener 1 into the tissue 16. After deployment, the crown 12 remains at the surface 18 of the tissue 16, and does not penetrate into the tissue 16. The prongs are preferably in the range of 3 to 7 mm in length so that after deployment, the barbs 13 are located in the fascia layer 21. Collagen fibers 22 in the fascia layer 21 entangle the barbs and thus promote the immobilization of the fastener 1 in the tissue 16. With the fastener 1 deployed as just explained, the surgical material 20 is pinned to the surface 18. In the case that the surgical material 20 has a mesh structure, as shown in FIG. 2, each of the prongs 11 pass through a different space in the mesh so that fibers 15 in the mesh pass under the crown between the prongs 11. The mesh 20 is thus prevented from slipping over the crown 12 and being released from the tissue surface 18.

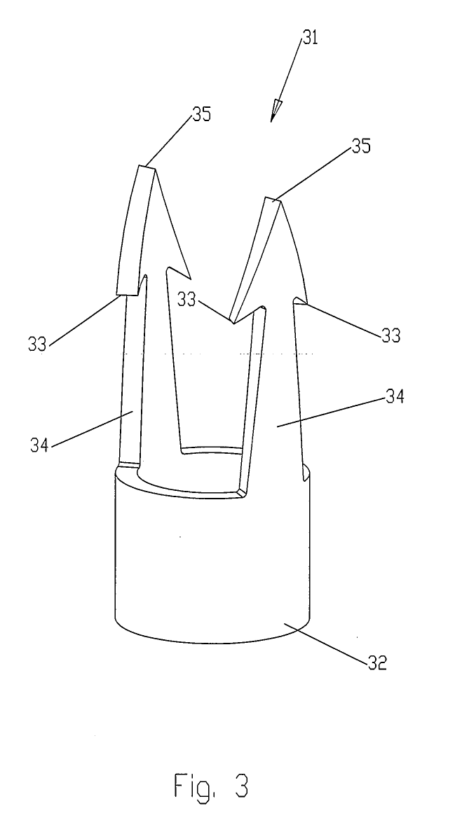

FIG. 3 shows a surgical fastener 31 in accordance with a second embodiment of the invention. The fastener 31 has a crown 32 in the form of a hollow cylinder. Two prongs 34 extend from the crown 32 parallel to the longitudinal axis of the crown 34. Each of the prongs 34 terminates in a pointed tip 35. Each prong 34 is also provided with a pair of barbs 33. Each barb 33 is pointed towards the crown 32 and away from the tip 35.

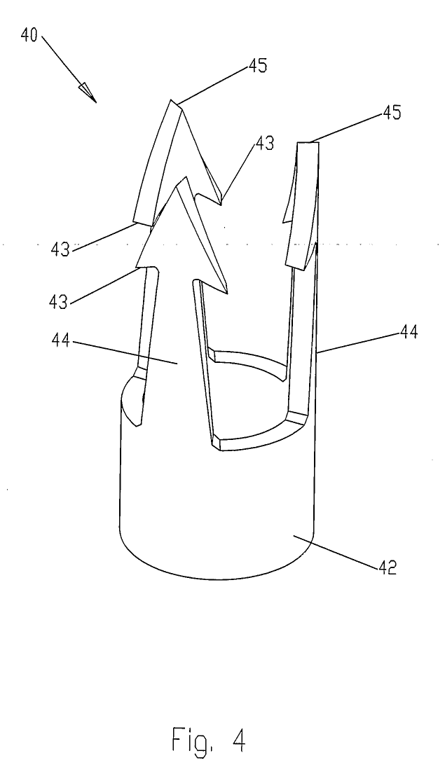

FIG. 4 shows a surgical fastener 40 in accordance with a third embodiment of the invention. The fastener 40 has a crown 42 in the form of a hollow cylinder. Three prongs 44 extend from the crown 42 parallel to the longitudinal axis of the crown 42. Each of the prongs 44 terminates in a pointed tip 45. Each prong is also provided with one or more barbs 43. Each barb 43 is pointed towards the crown 42 and away from the tip 45.

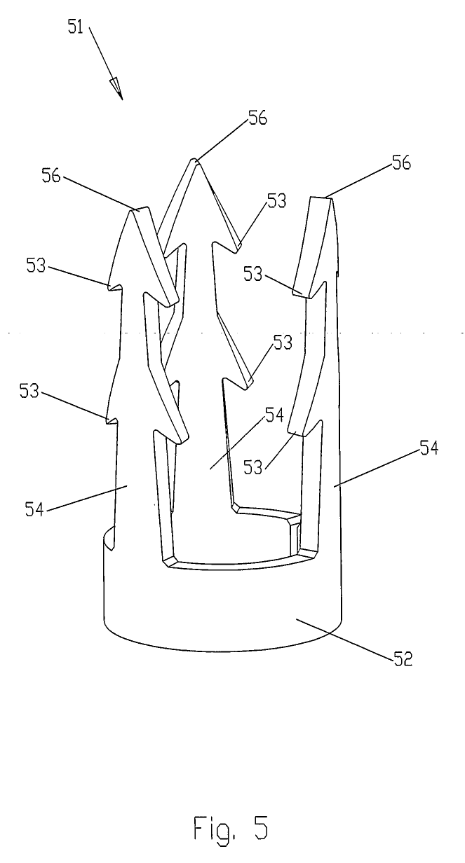

FIG. 5 shows another surgical fastener 51 of the invention. The fastener 51 has a crown 52 in the form of a hollow cylinder. Three prongs 54 extend from the crown 52 parallel to the longitudinal axis of the crown 52. Each of the prongs 54 terminates in a pointed tip 56. Each prong 54 is also provided with two pairs of barbs 53. Each barb 53 is pointed towards the crown 52 and away from the tip 56.

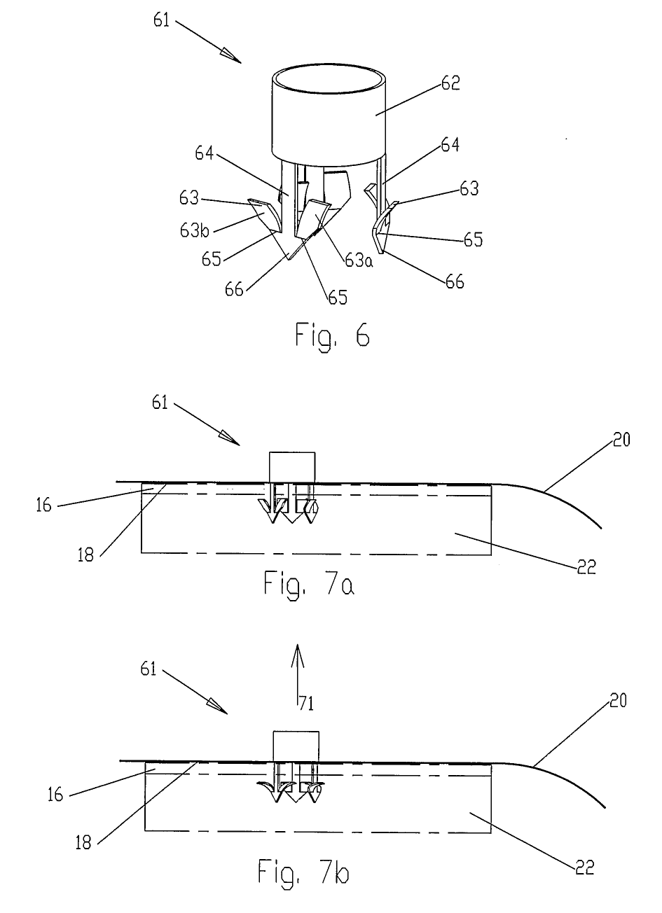

FIG. 6 shows a surgical fastener 61 in accordance with yet another embodiment of the invention. The fastener 61 has a crown 62 in the form of a hollow cylinder. Three prongs 64 extend from the crown 62 parallel to the longitudinal axis of the crown 62. Each of the prongs 64 terminates in a pointed tip 66. Each prong 64 is provided with one or more barbs 63. Each barb 63 is pointed towards the crown 62 and away from the tip 66. In this embodiment, each barb 63 is attached to the prong 64 at an integral hinge 65. In the in the undeployed configuration shown in FIG. 6, each barb 63 forms a small dihedral angle about the hinge 65, so that the barbs 63 slightly extend out of the plane of the rest of the prong 64. In the embodiment of FIG. 6, the two barbs 63 on a prong 64 extend from the plane of the prong in different directions, with one barb (for example the barb 63a) extending radially inward and the other barb (for example the barb 63b) extending radially outward.

FIG. 7 shows fastener 61 after deployment. In FIG. 7a, the fastener 61 has been deployed at the surface 18 of a body tissue 16, as explained above in reference to FIG. 2b. The crown 62 remains on the surface 18 and does not penetrate into the tissue 16. The surgical material 20 has been pinned to the surface 18 by the fastener 61 between the crown 62 and the surface 18. In the case that the surgical material 20 has a mesh structure, each of the prongs 64 passes through a different space in the mesh in order to prevent the mesh from slipping over the crown 62 and being released from the tissue surface 18.

As explained above, after deployment, the barbs 63 are located in the fascia layer 21. Collagen fibers 22 in the fascia layer 21 entangle the barbs and thus promote the immobilization of the fastener 61 in the tissue 16. Moreover, when a force is applied to the fastener 61 tending to pull the fastener out of the tissue 16, for example a force in the direction of the arrow 71, the collagen fibers 22 exert a force on the barbs 63 in the direction opposite to that of the arrow 71. In response to this force, the barbs 63 tend to rotate about the integral hinges 65. This increases the dihedral angle between each barb and the plane of the prong, as shown in FIG. 7b. In the configuration of FIG. 7b, the barbs 63 provide increased resistance to removal of the fastener 61 from the tissue 16, and thus increase the force needed to remove the fastener 61 from the tissue 16.

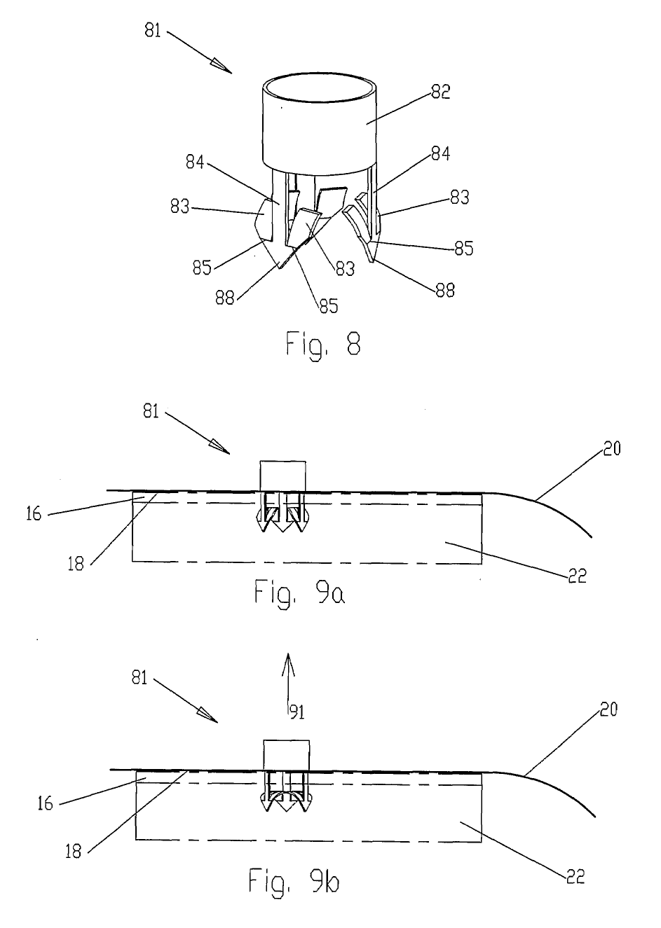

FIG. 8 shows a surgical fastener 81 in accordance with still another embodiment of the invention. The fastener 81 has a crown 82 in the form of a hollow cylinder. Three prongs 84 extend from the crown 82 parallel to the longitudinal axis of the crown 82. Each of the prongs 84 terminates in a pointed tip 88. Each prong 84 is provided with one or more barbs 83. Each barb 83 is pointed towards the crown 82 and away from the tip 88. In this embodiment, each barb 83 is attached to the prong 84 at an integral hinge 85. In the undeployed configuration shown in FIG. 8, each barb 83 forms a small dihedral angle about the hinge 85, so that the barbs 83 slightly extend out of the plane of the rest of the prong 84. In the embodiment of FIG. 8, the two barbs 83 on a prong 84 extend from the plane of the prong in the same direction, with both barbs extending radially inward.

FIG. 9 shows fastener 81 after deployment. In FIG. 9a, the fastener 81 has been deployed at the surface 18 of a body tissue 16, as explained above in reference to FIG. 2b. The crown 82 remains on the surface 18 and does not penetrate into the tissue 16. The surgical material 20 has been pinned to the surface 18 by the fastener 81 between the crown 82 and the surface 18. In the case that the surgical material 20 has a mesh structure, each of the prongs 84 passes through a different space in the mesh in order to prevent the mesh from slipping over the crown 82 and being released from the tissue surface 18.

As explained above, after deployment, the barbs 83 are located in the fascia layer 21. Collagen fibers 22 in the fascia layer 21 entangle the barbs 83 and thus promote the immobilization of the fastener 81 in the tissue 16. Moreover, when a force is applied to the fastener 81 tending to pull the fastener out of the tissue 16, for example a force in the direction of the arrow 91, the collagen fibers 22 exert a force on the barbs 83 in the direction opposite to that of the arrow 91. In response to this force, the barbs 83 tend to rotate about the integral hinges 85. This increases the dihedral angle between each barb and the plane of the prong, as shown in FIG. 9b. In the configuration of FIG. 9b, the barbs 83 provide increased resistance to removal of the fastener 81 from the tissue 16, and thus increase the force needed to remove the fastener 81 from the tissue 16.

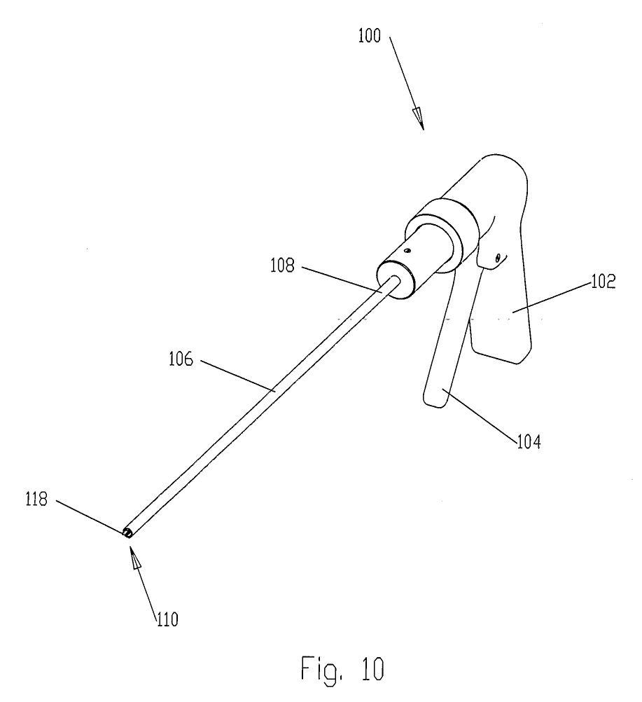

FIG. 10 shows a device 100 for deploying one or more surgical fasteners of the invention. The device 100 has a handle portion 102 containing an actuating mechanism including an actuating lever 104. A hollow slender shaft 106 extends from the handle portion 102 and stores one or more surgical fasteners of the invention. The shaft 106 has a proximal end 108 attached to the handle portion 102 and a distal end 110 end from which one or more of the surgical fasteners are sequentially ejected and deployed, as explained in detail below.

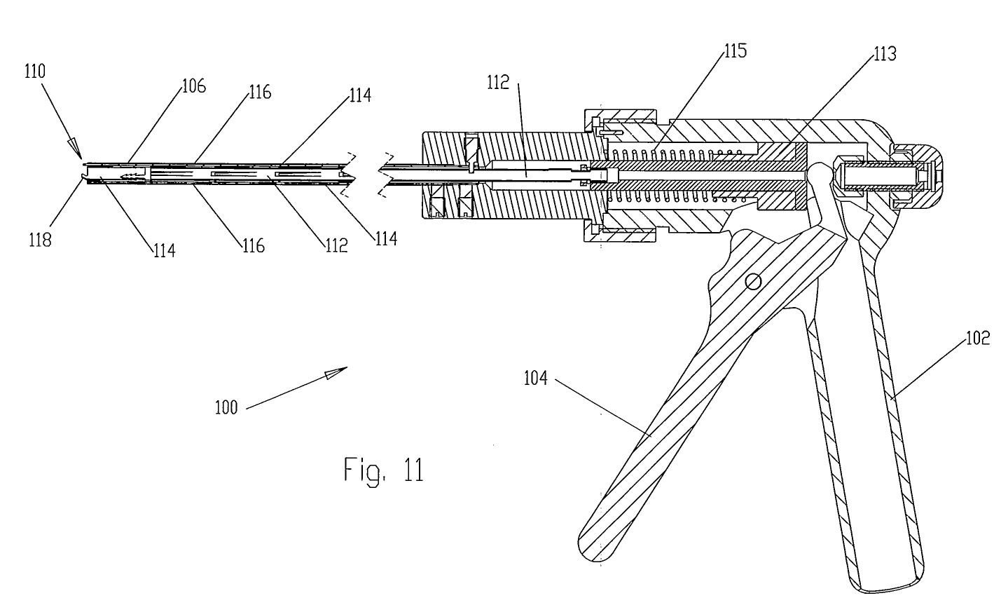

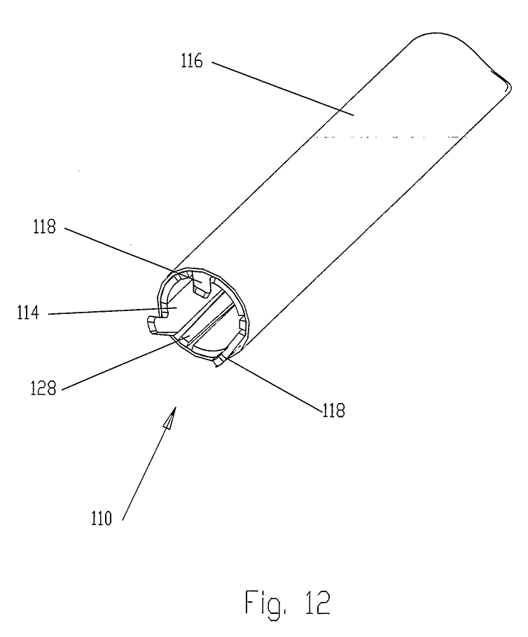

FIG. 11 shows a cross sectional view of the device 100. The shaft contains an inner sleeve 112, an outer sleeve 114 and an external sheath 116. The inner sleeve 112, the outer sleeve 114 and the external sheath 116 are hollow coaxial cylinders. Depressing the lever 104 causes an encasement 113 to move towards the distal end 100 of the shaft, while compressing a helical spring 115. The distally directed movement of the encasement 113 drives a rod 115 extending from the encasement 113 into the shaft 106. The rod pushes the inner sleeve 112 in a distal direction while the outer sleeve 114 remains stationary inside the shaft 106. As explained below, this causes a stack of fasteners inside the shaft 106 to move distally and causes the distal-most fastener in the stack to be ejected from the distal end 110 of the shaft. After a fastener has been released, the lever 104 is released. This causes the encasement 113, the rod 115 and the inner sleeve 112 to move in a proximal direction under the influence of the spring 115. As explained below, the inner sleeve and the outer sleeve cooperate to form a ratchet mechanism which prevents proximal movement of the stack of fasteners when the lever 104 is released.

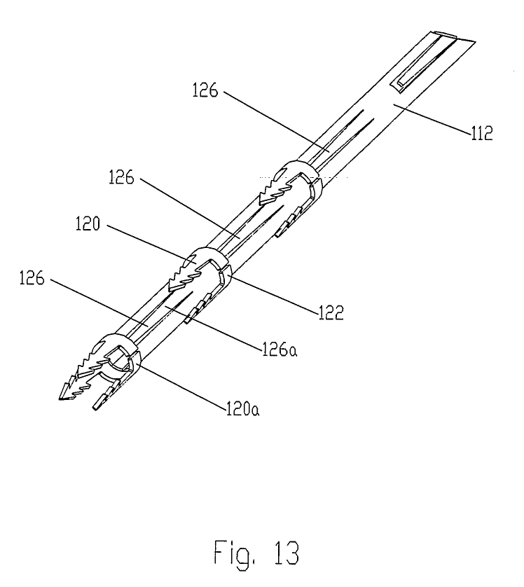

FIGS. 12 and 13 show the distal end 110 of the shaft 106 in greater detail. At the distal end 110 of the outer sleeve 114, the shaft 106 is provided with distally facing projections 118 that are designed to grasp a surgical mesh and to bring it to a desired location at a body site where it is to be pinned to a tissue surface by a surgical fastener of the invention, as described below. In order to facilitate grasping of the mesh, the projections 118 project at an acute angle to the distal rim of the sleeve.

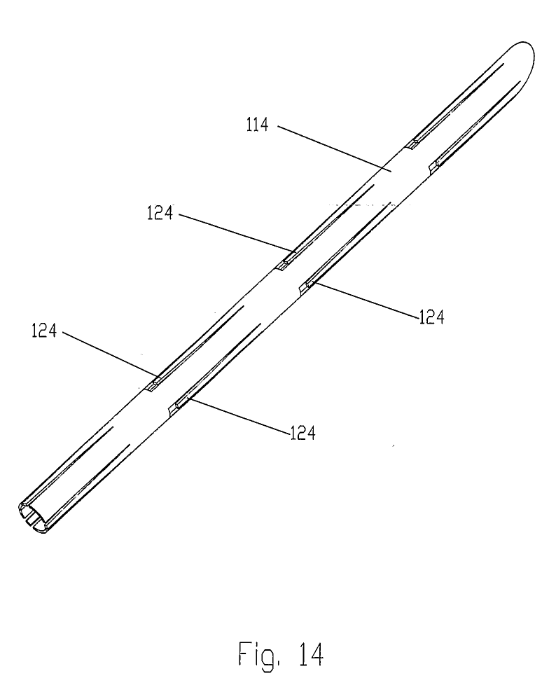

One or more surgical fasteners 120 of the invention are stacked within the shaft 106. As shown in FIG. 13, in this embodiment, the surgical fasteners have a crown shaped as a hollow cylinder 122, as shown in FIGS. 1 to 10. The inner sleeve 112 passes through the cylindrical crowns of the fasteners 120 inside the shaft 106 so that the axis of the shaft 106 is coaxial with the axes of the crowns 122. In a preferred embodiment the stack of fasteners is advanced by a ratchet mechanism. FIG. 14 shows the outer sleeve 114. The outer sleeve 114 is provided with radially inward facing projections 124 that prevent movement of the fasteners 120 inside the shaft in a proximal direction. The inner sleeve 112 is provided with radially outward facing projections 126. When, the inner sleeve 112 moves distally inside the shaft 106, each of the fasteners 120 experiences a force in the distal direction directly from the inner sleeve 112, via the projections 126. The projections 126 of the inner sleeve and the projections 124 of the outer sleeve act cooperatively to form a ratchet mechanism allowing the stack of fasteners to advance only distally inside the shaft 106 during each reciprocating movement of the inner sleeve relative to the outer sleeve. The distal-most projections 126a of the inner sleeve 112 pushing the distal-most fastener 120a distally and eject it from the shaft 106 during deployment. Radially inward facing projections 128 at the distal end of the outer sleeve 114 prevent inadvertent release of the distal-most fastener 120a from the distal end of the shaft 106. When the lever 104 is depressed, each fastener 120 in the shaft 106 is advanced one step distally, and the distal-most fastener is ejected from the distal end of the shaft.

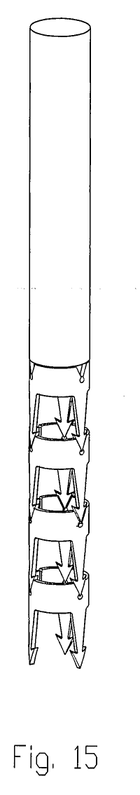

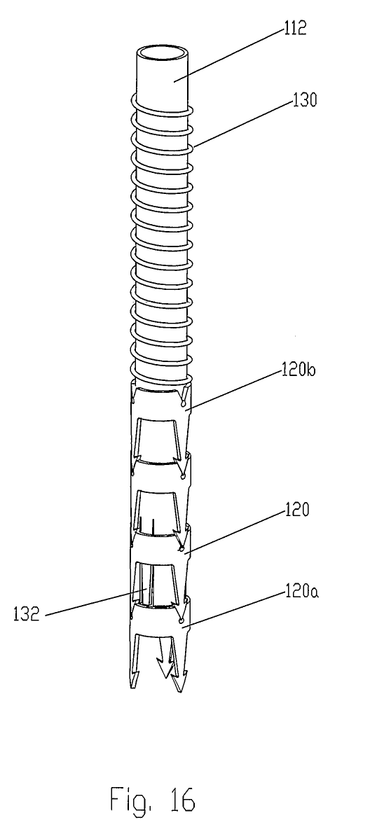

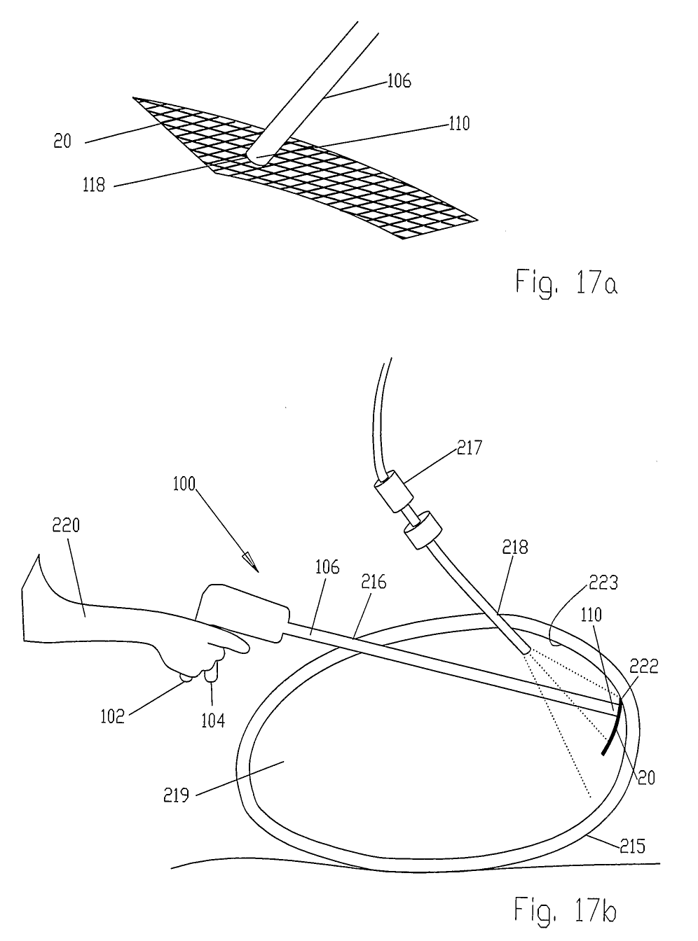

In an alternative arrangement, shown in FIG. 15, a distally directed force is applied by the actuating mechanism only to the proximal-most fastener in the stack of fasteners means by a rod or wire. This force is transferred from one fastener to the next in the stack of fasteners so that the entire stack of fasteners moves distally within the shaft 106. The distal-most fastener is thus ejected from the distal end of the shaft 106 and deployed. In yet another alternative arrangement shown in FIG. 16, a distally directed force is applied to the proximal-most fastener 120b in the stack of fasteners by a tensioned helical spring 130 that surrounds the inner sleeve 112. The inner sleeve 112 has at its end radially outward facing projections 132 that engage the crown of the distal-most fastener and eject it from the distal end of the shaft 106.

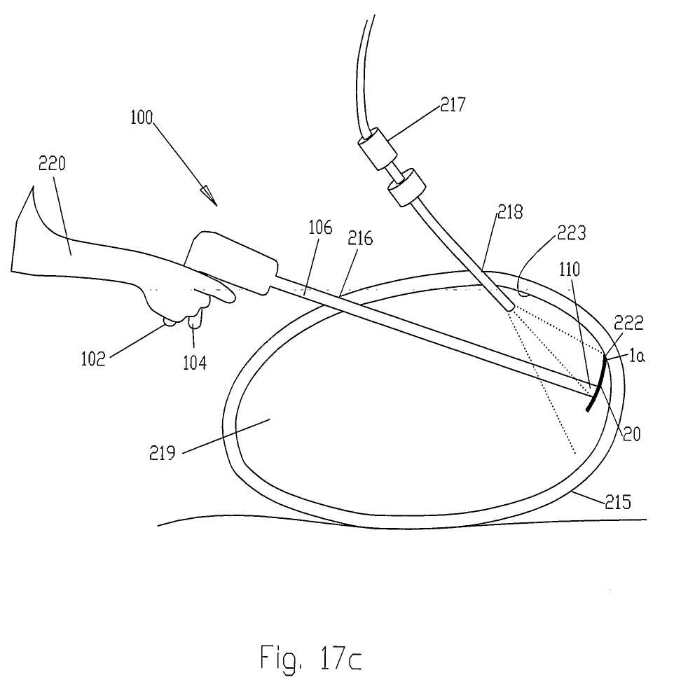

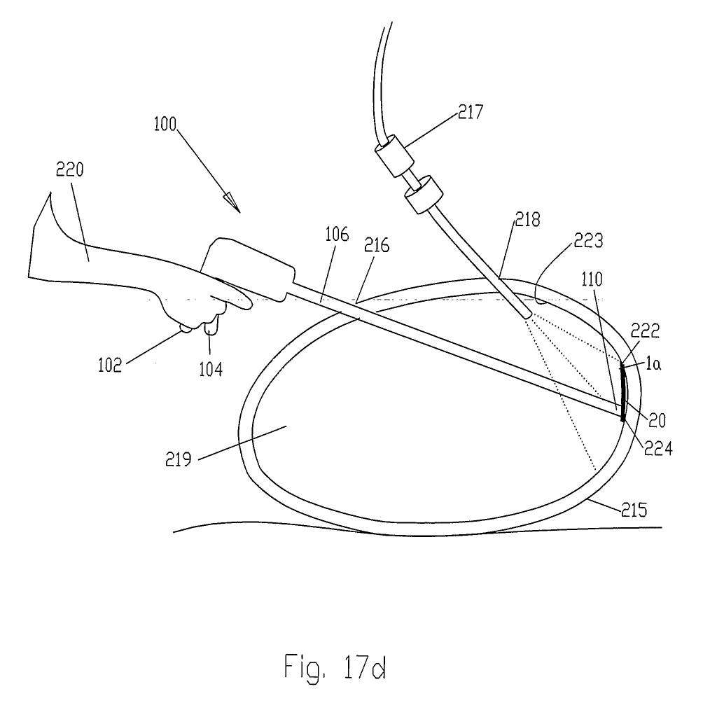

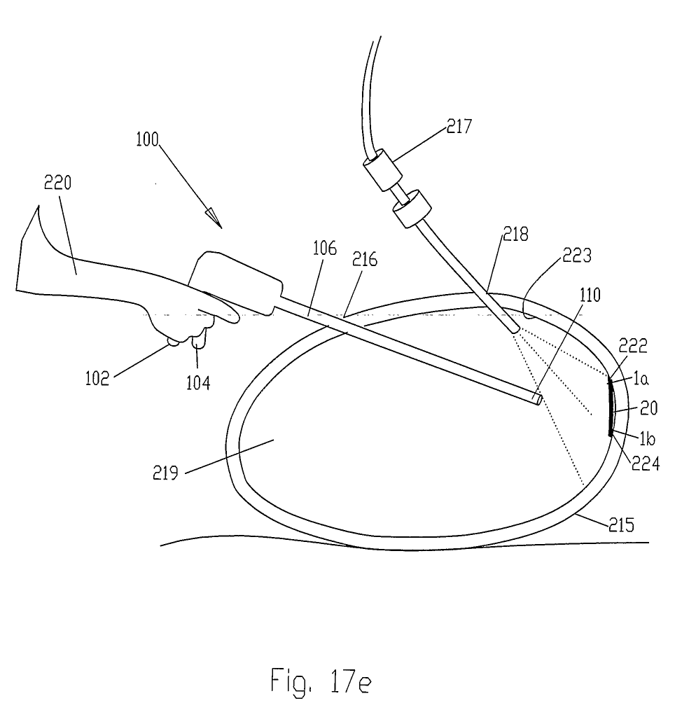

FIG. 17 depicts a surgical procedure in which a surgical fastening device of the invention, such as the fastening device 100, is used to attach a surgical mesh material to the internal surface of a body cavity. As shown in FIG. 17a, a piece of a surgical mesh material 20 is hooked onto the projections 118 at the end of the outer sleeve of the device 100. Then, as shown in FIG. 17b, the shaft 106 of the device 100 with the surgical mesh material hooked onto the distal end 110 of the shaft 106 is introduced into a body cavity 219 of a subject 215 through an incision at a first location 216 on the body surface. An endoscope 217 is introduced into the body cavity 219 through a second incision at a second location 218 on the body surface. The endoscope 217 illuminates the body cavity 219 containing the body tissue or tissues into which the fasteners 1 are to be inserted. The endoscope 217 is part of an imaging system that displays on a display screen (not shown), an image of the cavity 219, so as to allow a user 220 to observe the cavity 219 as the fasteners are deployed. The body cavity 219 may temporarily be expanded by inflation with a gas in order to enhance the maneuverability of the fastening device 100 and the endoscope 217 in the cavity 219. In this case, the external sheath 116 preferably has a smooth outer surface in order to decrease or prevent gas escape through the incision during the procedure.

In FIG. 17b, the distal end 110 of the fastening device 100 has been brought to a first location 222 on an internal surface 223 in the body cavity 219 where a first fastener is to be inserted. The user then squeezes the lever 104 against the handle portion 102 so as to actuate the actuating mechanism of the device 100, and eject a fastener from the distal end of the shaft into the tissue at the first location 222. As the fastener 1 is ejected from the distal end 110 of the shaft 106, the prongs 11 of the fastener 1 pass through the surgical mesh material 20, so that the surgical mesh material 20 becomes pinned to the first location 222 on the internal surface 223, as explained above in reference to FIG. 2.

FIG. 17c shows the surgical mesh material 20 after having been pinned to body tissue at the first location 222 by a fastener 1a. The user then detaches the distal end of the shaft 106 from the surgical mesh material 20. Disengagement of the sleeve from the mesh, after fixation of the mesh to the tissue is performed by pulling back the deployment device from the mesh. The user then hooks the distal end of the shaft 106 on at a new location on the mesh material 20, as shown in FIG. 17c. The mesh is engaged by these projections by slight pressure. The distal end 110 of the shaft is then brought to another location of the internal surface of the wall of the body cavity.

FIG. 17d shows the fastening device 100 after the distal end 110 has been brought to the second location 224 of the internal surface 223 in of the body cavity 219 where a second fastener is to be inserted into body tissue. The user then actuates the actuating mechanism of the device again to eject a second fastener 1b at the second location. As the second fastener 1b is ejected from the shaft, it grasps the mesh 20 in the vicinity of the distal end of the shaft, so as to pin the mesh 20 at the second location.

FIG. 17e shows the mesh 20 pinned at its ends at the first and second locations 222 and 224 by the fasteners 1a and 1b, respectively. This process may be repeated as required to securely attach the surgical material to the internal surface of the wall of the body cavity.

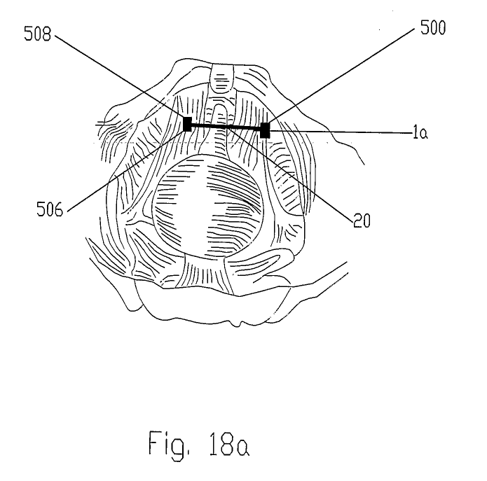

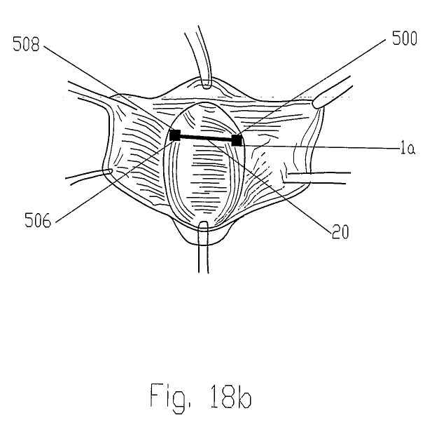

FIG. 18 shows use of the fastening device of the invention in a method of vaginal repair of stress incontinence. The procedure is shown in an abdominal view in FIG. 18a, and in a vaginal view in FIG. 18b. An incision 5 to 10 mm is made on the anterior vaginal wall over the urethra. A plane is then developed bilaterally between the vaginal wall and the urethopelvic ligament toward the attachment of this ligament to the arcuate ligament of the endopelvic fascia. A piece of surgical mesh material 20 is hooked onto the projections 118 at the end of the outer sleeve of a fastening device of the invention, such as the fastening device 100. The distal end of the fastening device, together with the hooked piece of mesh, is introduced through the incision towards the endopelvic fascia. A surgical fastener of the invention, such as the surgical fastener 1 is then ejected from the distal end of the fastening device so as to pin an end of the surgical material 20 at a first location 500 on the endopelvic fascia on one side. The fastening device is then detached from the mesh material 20 and removed through the vaginal incision. The surgical mesh material 20 is then hooked onto the projections 118 at another location on the mesh material and the shaft, together with the hooked mesh, is then reintroduced through the vaginal incision to the opposite endopelvic fascia and a second fastener 1b is ejected from the fastening device so as to pin the mesh material at a second location 508 on the second side of the endopelvic fascia. The distal end of the shaft is then detached from the mesh material so as to leave the mesh material stretched between the two endopelvic fascia. The device is then removed through the vaginal incision.

1. A surgical fastener comprising:
(a) a crown having an axis; and
(b) two or more prongs extending from the crown, each prong being parallel to the axis and each prong being provided with one or more barbs.

2. The surgical fastener according to claim 1 wherein the crown is a hollow cylinder having a longitudinal axis.

3. The surgical fastener according to claim 1 having three or more prongs.

4. The surgical fastener according to claim 1 wherein the barbs are attached to the prongs by a hinge.

5. The surgical fastener according to claim 4 wherein the hinge is an integral hinge.

6. The surgical fastener according to claim 4 wherein the barbs on each prong are rotated out of a plane determined by the prong.

7. The surgical fastener according to claim 4 wherein the barbs on each prong are staggered along the prongs.

8. The surgical fastener according to claim 1 wherein the prongs have a length from 3 mm to 7 mm.

9. A surgical fastening device configured to deploy one or more surgical fasteners, each surgical fastener comprising:
(i) a crown having an axis; and
(ii) two or more prongs extending from the crown, each prong being parallel to the axis and each prong being provided with one or more barbs;
the surgical fastening device comprising:
(a) a compartment configured to contain the one or more surgical fasteners; and
(b) an actuating mechanism configured to eject a fastener from the device without deforming the fastener.

10. The surgical fastening device according to claim 9 wherein the container has the shape of a cylindrical shaft having a distal end and a proximal end, a fastener being ejected by the actuating mechanism at the distal end of the shaft.

11. The surgical fastening device according to claim 10 wherein the actuating mechanism includes a ratchet mechanism permitting movement of the fasteners in the shaft in a distal direction and preventing movement of the fasteners in the shaft in a proximal direction.

12. The surgical fastening device according to claim 10 further comprising one or more extensions at the distal end of the shaft configured to engage a surgical mesh.

13. The surgical fastening device according to claim 10 wherein the crown of the one or more surgical fasteners is a hollow cylinder having a longitudinal axis.

14. The surgical fastening device according to claim 13 wherein the shaft contains:
(a) an inner sleeve configured to pass through the hollow cylindrical crown of the one or more surgical fasteners; and
(b) an outer sleeve configured to contain the one or more surgical fasteners.

15. The surgical fastening device according to claim 9 wherein the actuating mechanism comprise of a rod or wire applying a force to a stack of one or more fasteners when the actuating mechanism is actuating to eject a fastener from the device.

16. The surgical fastening device according to claim 15 wherein the force is applied to a proximal-most fastener in a stack of fasteners and the force is transmitted though the stack of fasteners to the distal-most fastener to eject the distal-most fastener.

17. The surgical fastening device according to claim 9, wherein an ejecting force is applied to the distal-most fastener through a tensioned spring.

18. The surgical fastening device according to claim 17 wherein the force is transmitted to a proximal-most fastener in a stack of fasteners and the force is transmitted though the stack of fasteners to the distal-most fastener to eject the distal-most fastener.

19. A system for surgical fastening comprising:
(a) a surgical fastening device configured to deploy one or more surgical fasteners, each surgical fastener comprising:
(i) a crown having an axis; and
(ii) two or more prongs extending from the crown, each prong being parallel to the axis and each prong being provided with one or more barbs;
the surgical fastening device comprising:
a compartment configured to contain the one or more surgical fasteners; and
an actuating mechanism configured to eject a fastener from the device without deforming the fastener; and
(b) One or more surgical fasteners, each surgical fastener comprising:
(i) a crown having an axis; and
(ii) two or more prongs extending from the crown, each prong being parallel to the axis and each prong being provided with one or more barbs.

20. The surgical fastening system according to claim 19 further comprising surgical material.

21. The surgical fastening system according to claim 19 wherein the surgical material is a surgical mesh.

22. Use of the surgical system according to claim 19 in a surgical procedure.

23. The use according to claim 22 wherein the surgical system comprises a surgical material and the surgical procedure comprises pinning a piece of the surgical material to a tissue surface.

24. The use according to claim 23 wherein the surgical material is a surgical mesh.

25. The use according to claim 24 wherein the container is in the form of a cylindrical shaft having hooks at a distal end, and the surgical procedure comprises engaging the surgical mesh on the hooks prior to actuating the actuating mechanism.

26. The use according to claim 25 wherein the surgical procedure comprises
(a) engaging a first region of the surgical mesh on the hooks;
(b) delivering the distal end of the shaft to a first body site;
(c) actuating the actuating mechanism to pin the first region of the mesh to the first body cite;
(d) disengaging the distal end of the shaft from the first region of the mesh;
(e) engaging a second region of the mesh on the hooks;
(f) delivering the distal end of the shaft to a second body site;
(g) actuating the actuating mechanism to pin the second region of the mesh to the second body site; and
(h) disengaging the distal end of the shaft from the second region of the mesh.

27. The use according to claim 22 in a surgical procedure selected from the group comprising treatment of stress incontinence, treatment of inguinal hernia, treatment of pelvic organ prolapse.

28. A method for surgical fastening, comprising:
(a) providing a surgical fastening device configured to deploy one or more surgical fasteners, each surgical fastener comprising:
(i) a crown having an axis; and
(ii) two or more prongs extending from the crown, each prong being parallel to the axis and each prong being provided with one or more barbs;
the surgical fastening device comprising:
a. a compartment containing one or more of the surgical fasteners; and
b. an actuating mechanism configured to eject a fastener from the device without deforming the fastener;
(b) delivering at least a portion of the surgical fastening device to a body site; and
(c) actuating the actuating mechanism to eject a surgical fastener into a body tissue at the body site.

29. The method according to claim 28 comprising pinning a surgical material to a tissue surface.

30. The method according to claim 29 wherein the surgical material is a surgical mesh.

31. The method according to claim 30 wherein the container is in the form of a cylindrical shaft having hooks at a distal end, and the surgical procedure comprises
(a) engaging a first region of the surgical mesh on the hooks;
(b) delivering the distal end of the shaft to a first body site;
(c) actuating the actuating mechanism to pin the first region of the mesh to the first body cite;
(d) disengaging the distal end of the shaft from the first region of the mesh;
(e) engaging a second region of the mesh on the hooks;
(f) delivering the distal end of the shaft to a second body site;
(g) actuating the actuating mechanism to pin the second region of the mesh to the second body site; and
(h) disengaging the distal end of the shaft from the second region of the mesh.

32. The method of surgical fastening according to claim 28 for use in a surgical procedure selected from the group comprising treatment of stress incontinence, treatment of inguinal hernia, treatment of pelvic organ prolapse.
